(19) United States
(12) Patent Application Publication
(10) Pub. No.: US 2024/0050628 A1
(43) Pub. Date: Feb. 15, 2024

(54) INJECTABLE PHOTOCROSSLINKED HYALURONIC ACID HYDROGELS, PRODUCTION METHOD THEREOF AND THEIR USE FOR THE TREATMENT OF OSTEOARTHRITIS

(71) Applicant: VSY BIYOTEKNOLOJI VE ILAC SANAYI ANONIM SIRKETI, ISTANBUL (TR)

(72) Inventors: Faruk OYTUN, Istanbul (TR); Mustafa CIFTCI, Istanbul (TR); Busra Gizem KAYA, Istanbul (TR)

(73) Assignee: VSY BIYOTEKNOLOJI VE ILAC SANAYI ANONIM SIRKETI, ISTANBUL (TR)

(21) Appl. No.: 18/269,760

(22) PCT Filed: Dec. 28, 2021
(86) PCT No.: PCT/TR2021/051565

§ 371 Date: Jun. 27, 2023

(30) Foreign Application Priority Data
Dec. 29, 2020 (TR) 2020/22151

(51) Int. Cl. A61L 27/52; C08B 37/08; C08J 3/075; C08L 5/08; A61L 27/20

(52) U.S. Cl. CPC A61L 27/52; C08B 37/0072; C08J 3/075; C08L 5/08; A61L 27/20; C08J 2305/08; A61L 2430/06; A61L 2400/06

(57) ABSTRACT

An injectable viscoelastic hydrogel formulation comprising photocrosslinked hyaluronic acid in order to relieve pain caused by osteoarthritis (OA), to support synovial fluid which has decreased activity, and to treat osteochondral lesions is provided. In the production of hyaluronic acid (HA) hydrogels, the crosslinking process is carried out via biocompatible camphorquinone instead of synthetic crosslinkers having toxic effects. Therefore, long process times in the purification process of synthetic crosslinkers are significantly reduced with the present invention.

### CROSS REFERENCE TO RELATED APPLICATIONS

This application is the national phase entry of International Application No. PCT/TR2021/051565, filed on Dec. 28, 2021, which is based upon and claims priority to Turkish Patent Application No. 2020/22151, filed on Dec. 29, 2020, the entire contents of which are incorporated herein by reference.

### FIELD OF INVENTION

The present invention relates to the development of an injectable viscoelastic hydrogel formulation comprising photocrosslinked hyaluronic acid in order to relieve pain caused by osteoarthritis (OA), to support synovial fluid which has decreased activity, and to treat osteochondral lesions.

### BACKGROUND

The joint can be defined as the location where two bones come together in movable parts of the body such as arms, legs, feet, wrists, and the ends of these bones are covered with a protective tissue called cartilage. Cartilage is a durable and slippery tissue which allows joint movement to occur almost without friction. Synovial fluid, which provides lubricity between bone ends and cartilage, is comprised of Hyaluronic Acid (HA) that is naturally present in the body. The viscosity, lubrication and shock absorbing properties of the said synovial fluid decrease over time due to mechanical effects and the cartilages begin to rub against each other and break down. As the cartilage wears off and breaks down over time, the friction between the bones increases and a joint disease called Osteoarthritis (OA) begins and progresses with the increase in friction.

Osteoarthritis (OA) is the most common, chronic (long-term) joint disease affecting millions of people worldwide. OA is also called degenerative joint disease, degenerative arthritis, and wear and tear arthritis. OA affects the entire joint, as well as cartilage breakdown. It causes changes in the bone structure and the deterioration of the connective tissues that hold the joint together and connect the muscles to the bone; and it also causes inflammation of the joint capsule. Even though OA is usually seen in old ages, it can sometimes be seen in young people. OA symptoms develop over time and they worsen as it progresses. Symptoms of OA include pain, stiffness, tenderness, loss of flexibility, crepitus, osteophyte formation, and swelling.

Even though damage of the joints cannot be recovered; exercising, being at a healthy weight and several treatments that are applied can slow the progression of the disease and help relieve pain and improve joint functions.

One of the methods that can help relieve OA symptoms, especially pain, is the use of medication. The commonly used drugs and their effects are shown below:
Acetaminophen: It is known that acetaminophen (Tylenol and others) helps patients with osteoarthritis suffering from mild to moderate pain. Taking acetaminophen more than the recommended dose may cause liver damage.
Non-steroidal anti-inflammatory drugs (NSAIDs): NSAIDs such as Advil (ibuprofen) and Aleve (naproxen) reduce swelling as well as pain.
Duloxetine (Cymbalta): This drug which is generally used as an antidepressant is also used to treat many chronic pains, including osteoarthritis pain.

In addition to these, surgical and other procedures that can be used to relieve the symptoms of OA are listed below:
Cortisone injections: In corticosteroid drug injections, the area around the joint is checked, and then the drug is injected by inserting a needle into the space in the joint. However, the number of cortisone injections that can be administered annually is generally limited to three or four. This is because the drug worsens joint damage over time.
Viscoelastic injections: Injections of HA, which is a component found naturally in the synovial fluid, exhibit a buffering effect on the knee and alleviate the severity of pain.
Joint replacement: In joint replacement surgery (arthroplasty), the damaged joint surfaces are replaced with plastic and metal parts. However, this method involves surgical risks related with the infections. In addition, artificial joints can also wear out and loosen over time and may need to be replaced.

Today, the most preferred method among the methods of the treatment is to make viscoelastic injections to the problematic area. The most widely used of these injection types is HA injections. People with OA have low amounts of HA in their joint fluid. In the knee with OA, the concentration of HA is approximately half of the normal level. HA is a gel-like substance naturally existing in the synovial fluid surrounding the joints. The said substance provides lubrication and cushioning, acts as a shock absorber, and helps the bones move smoothly by preventing the bones from wearing each other off. Furthermore, it supports the synovial fluid to ensure the healing of intra-articular wounds in pathological conditions. In addition, it improves the physiological environment of the osteoarthritic joint and increases its mobility by regaining its viscoelasticity.

Changes in the viscous and elastic properties of HA decrease the joint's ability to withstand tensile and shear forces. HA injections can help recover the normal levels of synovial fluid. These injections not only lubricate the joint, they can also reduce inflammation and protect the cartilage from further wear and tear.

The injections are made in weekly periods by the doctor or healthcare professional. The number of injections to be made may vary depending on the HA concentration.

Before making the injection, the doctor can perform aspiration by taking some of the fluid from the knee joint with a needle in order to reduce swelling.

The hyaluronic acid which is not crosslinked (Linear HA) has several disadvantages such as its short term effect and its inability to create volume. HA is subjected to crosslinking processes with crosslinking agents such as ethyldimethylaminopropyl carbodiimide (EDC), butanediol diglycidylether (BDDE), glutaraldehyde (GTA) or divinyl sulfone (DVS) in order to improve its viscoelastic properties and increase its permanence. Crosslinking reaction is an intramolecular and intermolecular esterification reaction that is performed using several reagents. It has been envisaged in this technique, the residence time of the HA formulation in the joint can be increased. Therefore, while linear HA injections are usually applied in 3-5 sessions, this process can be performed in a single session in viscoelastics comprising cross-linked HA. This makes cross-linked HA injections, which have a longer residence time in the body and higher mechanical strength, to be preferred more.

The physical, chemical and biological properties of HA change significantly by increasing the degree of crosslinking. The gel suspension of crosslinked HA has different rheological properties than the linear HA. The rheological properties of cross-linked HA gels exhibit superior properties than non-crosslinked HA at the same concentration.

In the state of the art, in cases when HA is applied to the joint area, it changes the rheology of the synovial fluid, provides a quick relief in mobility and relieves pain. However, the effect of the conventionally used Linear HA is temporary, because HA can only stay for 72 hours within the joint before it is absorbed or metabolized. As well as this situation does not prolong the patient's comfort period sufficiently, the damaged articular cartilage cannot be healed since the main problem is not improved.

U.S. Pat. No. 8,323,617B2, an application known in the state of the art, discloses that HA formulations crosslinked with carbodiimide derivative crosslinking agents are used in the treatment of OA. Since the formulations obtained by the direct crosslinking method require harsh reaction conditions, there is a possibility that toxic by-products may be generated; and since the crosslinking agents that are used are cytotoxic, they may not be considered suitable for hydrogel design.

European patent document no EP1443945B1, an application known in the state of the art, discloses synergistic effect of sodium hyaluronate and chondroitin sulfate mixture on the lubrication and regeneration of articular cartilage damaged by stage I and stage II osteoarthritis of human joints. In this patent application, the combination of linear HA and chondroitin sulfate was used in the treatment of osteoarthritis. It is believed that it is unlikely that the viscoelastic product to be used in such formulations where crosslinking agent is not used will provide comfort to the patient in a single session.

An article published by Falcone, S. J. et. al., an application known in the state of the art, discloses the use of hyaluronic acid in biomedical applications, its physical properties, rheological properties and differences of cross-linked HA from linear HA. Falcone, S. J., Palmeri, D., & Berg, R. A. (2006): “Biomedical Applications of Hyaluronic Acid”; Polysaccharides for Drug Delivery and Pharmaceutical Applications, 155-174. doi:10.1021/bk-2006-0934.ch008.

### SUMMARY

The objective of the present invention is to develop an injectable viscoelastic hydrogel formulation comprising photocrosslinked hyaluronic acid in order to relieve pain caused by osteoarthritis (OA), to support synovial fluid which has decreased activity, and to treat osteochondral lesions.

Another objective of the present invention is to ensure that the photocrosslinked HA viscoelastic gel can be used in human joints suffering from osteoarthritis (application site including knee, shoulder, sacroiliac, hip, ankle, elbow, interphalangeal and wrist joints) for lubrication of the joint and self-regeneration of the cartilage.

Another objective of the invention is to significantly reduce the purification processes that are required for the removal of synthetic crosslinkers, which can exhibit toxic properties, from the formulation and that require long periods in the production process.

A further objective of the invention is to not use synthetic crosslinkers with toxic effects, but to carry out the crosslinking process through camphorquinone, which is widely used in many bio-applications, especially in dental applications.

Yet another objective of the present invention is to adapt photo-initiated polymerization methods, which meet many economic and ecological expectations, to the production of hyaluronic acid hydrogels.

### BRIEF DESCRIPTION OF THE DRAWINGS

An injectable photocrosslinked hyaluronic acid hydrogel developed in order to fulfil the objectives of the present invention is illustrated in the accompanying figures, in which:

FIGS. 1A-1B Illustration of the reaction of obtaining initiator radicals using second type of photo-initiators. (FIG. 1A: Illustration of general reaction, FIG. 1B: Benzophenone example).

FIG. 2—Illustration of the synthesis of acrylated hyaluronic acid monomer in single step.

FIG. 3—Illustration of the production of HA-based hydrogels via camphorquinone with the effect of light without crosslinker.

### DETAILED DESCRIPTION OF THE INVENTION

The components shown in the FIGS. are each given reference characters as follows:
FB. Photo-initiator,
[FB]*. Excited state photo-initiator
YB. Co-initiator,
YB^{•}. Activated co-initiator,
hv. Light,
TEA. Triethylamine, and
HA-TH. Hyaluronic Acid Based Hydrogel.

A production method of an injectable viscoelastic hydrogel formulation comprising photocrosslinked hyaluronic acid in order to relieve the pain caused by osteoarthritis (OA) and to support synovial fluid which has decreased activity comprises the steps of:
Production of acrylated hyaluronic acid monomer:
   Dissolving hyaluronic acid (HA) by slowly adding it into deionized water,
   Dissolving glycidyl acrylate and triethylamine in dimethylformamide,
   Combining these two solutions and mixing for 3 days at room temperature,
   Precipitating the mixture in acetone weighing up to 20 times its own weight and filtering it under low pressure,
   Removing the impurities of the obtained solid with deionized water,
   Drying the mixture by freeze drying method, and
   Obtaining acrylated HA monomer.
Production of photocrosslinked hyaluronic acid hydrogels:
   Dissolving the obtained acrylated HA monomer by slowly adding it into deionized water,
   Adding camphorquinone to this HA mixture and mixing it homogeneously,
   Illuminating the mixture under visible light and leaving it for cross-linking reaction,
   Neutralizing the gel in the buffer solution after the reaction until the pH value becomes stable,
   Filtering after pH adjustment, and allowing the gel to swell upon adding a new buffer solution,
   Then, bringing the obtained gel to a particle size of 50-300 microns,
   Adding non-crosslinked HA (Linear HA) into the photocrosslinked HA gel to facilitate its extrusion,
   Adjusting the pH of the final mixture to 7 and filling it into the syringes by applying vacuum, and
   Applying vapor sterilization process to the syringes after filling process.

In one embodiment of the invention, 5.0 mmol (2.0 grams) of HA is slowly added into 100 mL of deionized water and dissolved therein within the scope of production of acrylated hyaluronic acid monomer.

In one embodiment of the invention, 250 mmol (32.2 grams) of glycidyl acrylate and 250 mmol (25.3 grams) of triethylamine are dissolved in 100 mL of dimethylformamide.

In one embodiment of the invention, 5.0 mmol (2.0 g) of acrylated HA monomer is slowly added into 100 ml of deionized water and dissolved therein within the scope of production of photocrosslinked HA hydrogels.

In one embodiment of the invention, 0.05-1% by weight of camphorquinone is added to the HA mixture and mixed homogenously.

In one embodiment of the invention, the mixture of camphorquinone and HA is illuminated under visible light for 30-240 minutes at room temperature and left for crosslinking reaction.

In one embodiment of the invention, after the crosslinking reaction, the gel is neutralized with 0.1 M of hydrochloric acid (HCl) in phosphate buffer solution (PBS) until the pH is 6.8-7.4.

The viscoelastic gel formulation obtained within the scope of the invention is used for the treatment of osteochondral lesions caused by osteoarthritis (OA). The viscoelastic gel formulation obtained within the scope of the invention is used in intra-articular injection applications at concentrations of 10-25 mg/ml.

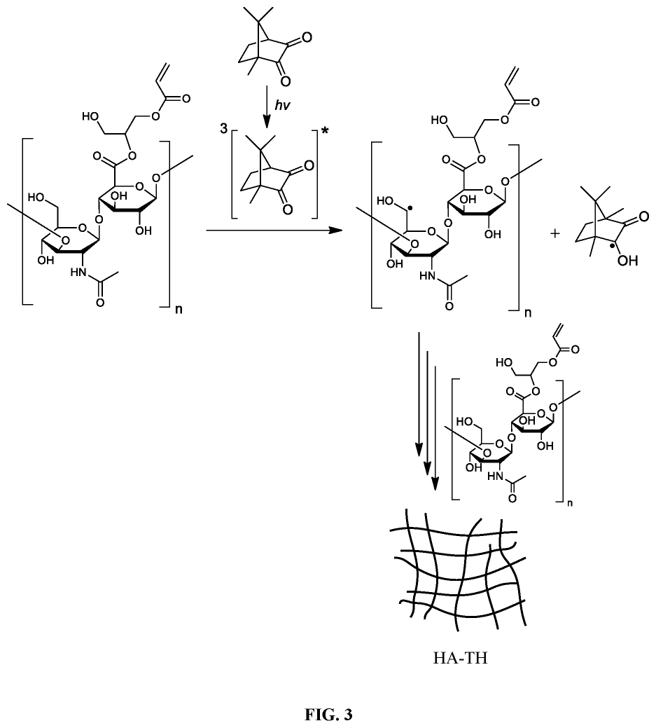

Within the scope of the invention, initially, the hyaluronic acid macromonomer comprising acrylate groups is synthesized in a simple way in a single step (FIG. 2). When the obtained acrylated hyaluronic acid monomer and camphorquinone are dissolved in water and illuminated under visible light, HA-based hydrogels are obtained (FIG. 3). In the first step of the process, the camphorquinone is excited with the effect of light and then the excited camphorquinone forms initiator radicals by abstracting a hydrogen atom (a hydrogen) from the carbon atom (a carbon) neighboring the hydroxyl group on the hyaluronic acid. These radicals that are formed initiate the free radical polymerization of acrylate groups in the HA monomer, enabling the formation of HA-based hydrogels. Since the said HA monomers comprise more than one acrylate group, gels are obtained easily without any crosslinkers. In other words, the acrylated HA monomer acts both as a conventional monomer and as a crosslinker. In addition, it is predicted that gels with different crosslinking densities can be obtained by changing the camphorquinone concentration. Therefore, gels with different crosslink densities that are needed can be obtained in a simple way.

The viscoelastic gel formulation developed within the scope of the invention, unlike conventional methods, enables to obtain photocrosslinked production of hydrogels comprising HA without any crosslinkers. Acrylated hyaluronic acid and camphorquinone, which are the two main components of the formulation, can be easily obtained as a result of illumination under visible light. Therefore, the need for crosslinking agents such as EDC (1-ethyl-3-(3-dimethylaminopropyl)carbodiimide hydrochloride), BDDE (1,4-butanediol diglycidyl ether), GTA (glutaraldehyde), and DVS (divinyl sulfone) in conventional applications is eliminated. As a result of this, the purification processes that require long time and effort, namely the removal processes of these crosslinkers that are used from the final product, will be significantly reduced. For example, the amount of crosslinker BDDE must be less than 2 ppm in the final product, as per the requirements set by the FDA. This may lead to the utilization of multiple washing processes and special purification methods in the purification step. On the other hand, in the method used in the development of the viscoelastic gel formulation developed within the scope of the invention, such crosslinkers with toxic effects will not be used, and the crosslinking will be carried out through camphorquinone, which is widely used in many bio-applications, especially in dental applications.

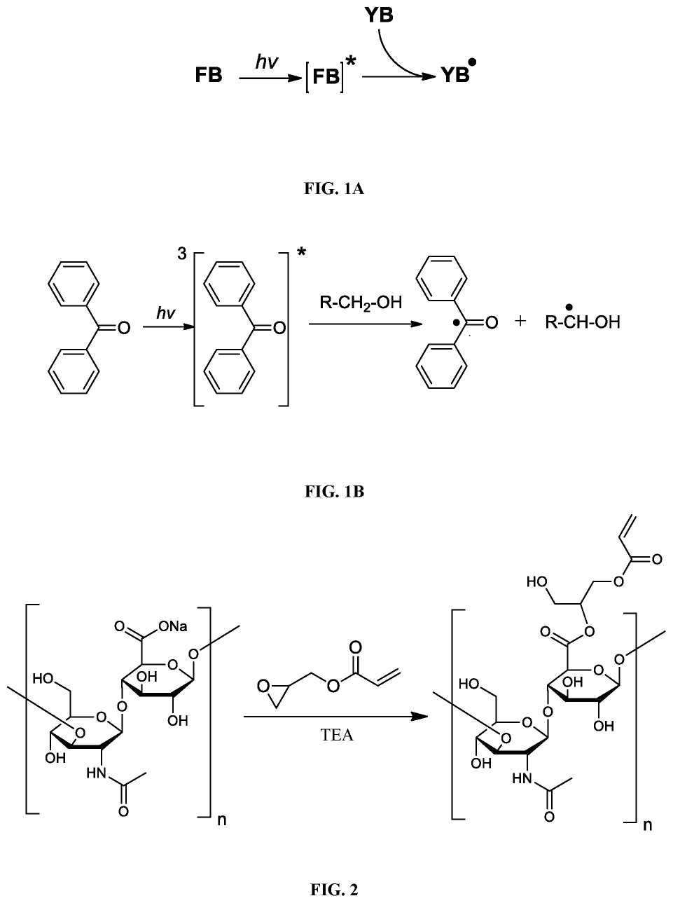

Camphorquinone is a second type photo-initiator which absorbs visible light between 400-500 nm (maximum 468 nm). In general, in the second type of photo-initiators, the initiator becomes excited by the effect of light. However, since the excitation energies of these excited states are lower than the bond dissociation energies, they cannot directly form initiator radicals. Instead, excited types transfer energy with a second type called co-initiator, abstract hydrogen from co-initiators and generate radicals on the co-initiators (FIGS. 1A-1B). In the second type photo-initiators, alcohol, amine, ether and thiol molecules, which have hydrogen donor properties, are used as co-initiators.

Even though there are different types of second type photo-initiators in the state of the art, camphorquinone has a special importance among these initiators as it exhibits many superior properties. Since camphorquinone can be excited in the visible region of the light (low energy), it both provides energy saving and it is suitable for biological applications since UV region light having harmful effects is not used. Hence camphorquinone is a second type photo-initiator which is most widely used in many bio-applications, especially in dental filling materials. The most important feature of camphorquinone that makes it widely used in such biological applications is its biocompatibility. In addition, camphorquinone shows negative effects in the body only at high concentrations. For example, see M. C. Chang, L. D. Lin, M. T. Wu et al., Effects of camphorquinone on cytotoxicity, cell cycle regulation and prostaglandin E2 production of dental pulp cells: role of ROS, ATM/Chk2, MEK/ERK and hemeoxygenase-1, PLoS One, vol. 10, no. 12, article e0143663, 2015, and Geurtsen W. Biocompatibility of resin-modified filling materials. Crit Rev Oral Biol Med 2000; 11(3):333-55. Furthermore, due to the nature of the reaction, since camphorquinone will not bind to the gel structure that will be formed, it can be easily removed. This will considerably shorten the purification process.

Within the scope of the invention, the photo-initiated polymerization method, which is a method that meets many economic and ecological expectations, is adapted to the production of hyaluronic acid hydrogels. Therefore, the advantages of photo-initiated polymerization methods such as high polymerization rate at low temperatures (including room temperature), low energy consumption, polymerization in solvent-free environment, and temporal/spatial control facilitate the use of this technique in industrial production.

The advantages of the production method of gel formulation according to the present invention can be listed as follows:
Easily obtaining hydrogels by means of illuminating the two main components of the formulation, namely acrylated hyaluronic acid and camphorquinone, under visible light,
Obtaining hydrogels comprising HA in a photocrosslinked form without using any synthetic crosslinkers unlike conventional methods,
Significantly reducing the purification processes that are required for the removal of synthetic crosslinkers which can exhibit toxic properties from the formulation and that require long periods in the production process.
Facilitating the use of this technique in industrial production with the advantages of photo-initiated polymerization methods such as high polymerization rate at low temperatures (including room temperature), low energy consumption, polymerization in solvent-free environment, and temporal/spatial control by means of adapting photo-initiated polymerization methods to the production of hyaluronic acid hydrogels, and
Increasing the mechanical strength of the gel and its residence time in the joint by means of the photocrosslinked HA in the mixture.

What is claimed is:

1. A production method for a syringe comprising injectable viscoelastic hydrogel formulation comprising photocrosslinked hyaluronic acid hydrogel that relieves pain caused by osteoarthritis (OA) and to support synovial fluid which has decreased activity comprising the steps of:
production of acrylated hyaluronic acid monomer by
dissolving hyaluronic acid by slowly adding it into deionized water to form a first solution,
dissolving glycidyl acrylate and triethylamine in dimethylformamide to form a second solution,
combining the first and second solutions and mixing for 3 days at room temperature to form a mixture,
precipitating the mixture in acetone and filtering it under low pressure to provide an obtained solid,
removing impurities of the obtained solid with deionized water,
then drying the obtained solid by freeze drying method, obtaining the acrylated hyaluronic acid monomer;
production of photocrosslinked hyaluronic acid hydrogels by
dissolving the acrylated hyaluronic acid monomer by slowly adding it into deionized water to obtain a first acrylated hyaluronic acid mixture,
adding camphorquinone to this first acrylated hyaluronic acid mixture and mixing it homogeneously to obtain a second acrylated hyaluronic acid mixture,
illuminating the second acrylated hyaluronic acid mixture under visible light and leaving it for cross-linking reaction to obtain a gel,
neutralizing the gel in a buffer solution after the reaction until the pH value becomes stable,
filtering the gel after pH adjustment, and allowing the gel to swell upon adding a new buffer solution to obtain a swollen gel,
then, bringing the swollen gel to a particle size of 50-300 microns,
then adding non-crosslinked hyaluronic acid into the swollen gel having a particle size of 50-300 microns to facilitate its extrusion to provide the photocrosslinked hyaluronic acid hydrogel, and
production of the syringe comprising injectable viscoelastic hydrogel formulation comprising photocrosslinked hyaluronic acid hydrogel by
adjusting the pH of the photocrosslinked hyaluronic acid hydrogel to 7 and filling it into the syringes by applying vacuum,
applying vapor sterilization process to the syringe after filling.

2. The production method for the syringe comprising injectable viscoelastic hydrogel formulation comprising photocrosslinked hyaluronic acid hydrogel according to claim 1, wherein when dissolving the hyaluronic acid, 5.0 mmol (2.0 grams) of hyaluronic acid is added into 100 mL of the deionized water.

3. The production method for the syringe comprising injectable viscoelastic hydrogel formulation comprising photocrosslinked hyaluronic acid hydrogel according to claim 1, wherein when dissolving glycidyl acrylate and triethylamine in the dimethylformamide, 250 mmol (32.2 grams) of glycidyl acrylate and 250 mmol (25.3 grams) of triethylamine are dissolved in 100 mL of the dimethylformamide.

4. The production method for the syringe comprising injectable viscoelastic hydrogel formulation comprising photocrosslinked hyaluronic acid hydrogel according to claim 1, wherein when dissolving the acrylated hyaluronic acid monomer in deionized water, 5.0 mmol (2.0 g) of acrylated hyaluronic acid monomer is slowly added into 100 ml of the deionized water and dissolved therein.

5. The production method for the syringe comprising injectable viscoelastic hydrogel formulation comprising photocrosslinked hyaluronic acid hydrogel according to claim 1, wherein when adding camphorquinone to the first acrylated hyaluronic acid mixture, 0.05-1% by weight of camphorquinone is added to the first hyaluronic acid mixture.

6. The production method for the syringe comprising injectable viscoelastic hydrogel formulation comprising photocrosslinked hyaluronic acid hydrogel according to claim 1, wherein when illuminating the second acrylated hyaluronic acid mixture, the second hyaluronic acid mixture of is illuminated under visible light for 30-240 minutes at room temperature and left for crosslinking reaction.

7. The production method for the syringe comprising injectable viscoelastic hydrogel formulation comprising photocrosslinked hyaluronic acid hydrogel according to claim 1, wherein when neutralizing gel, the gel is neutralized with 0.1 M of hydrochloric acid (HCl) in phosphate buffer solution (PBS) until the pH is 6.8-7.4.

8. A syringe comprising the injectable viscoelastic gel formulation comprising photocrosslinked hyaluronic acid hydrogel, which is obtained with a production method according to claim 1 and is configured for the treatment of osteochondral lesions caused by osteoarthritis (OA).

9. The syringe comprising the injectable viscoelastic gel formulation comprising photocrosslinked hyaluronic acid hydrogel according to claim 8, wherein the photocrosslinked hyaluronic acid hydrogel is at a concentration of 10-25 mg/ml.

10. The syringe comprising the injectable viscoelastic gel formulation comprising photocrosslinked hyaluronic acid hydrogel according to claim 8, wherein when dissolving the hyaluronic acid, 5.0 mmol (2.0 grams) of hyaluronic acid is added into 100 mL of the deionized water.

11. The syringe comprising the injectable viscoelastic gel formulation comprising photocrosslinked hyaluronic acid hydrogel according to claim 8, wherein when dissolving glycidyl acrylate and triethylamine in the dimethylformamide, 250 mmol (32.2 grams) of glycidyl acrylate and 250 mmol (25.3 grams) of triethylamine are dissolved in 100 mL of the dimethylformamide.

12. The syringe comprising the injectable viscoelastic gel formulation comprising photocrosslinked hyaluronic acid hydrogel according to claim 8, wherein when dissolving the acrylated hyaluronic acid monomer in deionized water, 5.0 mmol (2.0 g) of acrylated hyaluronic acid monomer is slowly added into 100 ml of the deionized water and dissolved therein.

13. The syringe comprising the injectable viscoelastic gel formulation comprising photocrosslinked hyaluronic acid hydrogel according to claim 8, wherein when adding camphorquinone to the first acrylated hyaluronic acid mixture, 0.05-1% by weight of camphorquinone is added to the first hyaluronic acid mixture.

14. The syringe comprising the injectable viscoelastic gel formulation comprising photocrosslinked hyaluronic acid hydrogel according to claim 8, wherein when illuminating the second acrylated hyaluronic acid mixture, the second hyaluronic acid mixture of is illuminated under visible light for 30-240 minutes at room temperature and left for crosslinking reaction.

15. The syringe comprising the injectable viscoelastic gel formulation comprising photocrosslinked hyaluronic acid hydrogel according to claim 8, wherein when neutralizing gel, the gel is neutralized with 0.1 M of hydrochloric acid (HCl) in phosphate buffer solution (PBS) until the pH is 6.8-7.4.

16. The syringe comprising the injectable viscoelastic gel formulation comprising photocrosslinked hyaluronic acid hydrogel according to claim 10, wherein the photocrosslinked hyaluronic acid hydrogel is at a concentration of 10-25 mg/ml.

17. The syringe comprising the injectable viscoelastic gel formulation comprising photocrosslinked hyaluronic acid hydrogel according to claim 11, wherein the photocrosslinked hyaluronic acid hydrogel is at a concentration of 10-25 mg/ml.

18. The syringe comprising the injectable viscoelastic gel formulation comprising photocrosslinked hyaluronic acid hydrogel according to claim 12, wherein the photocrosslinked hyaluronic acid hydrogel is at a concentration of 10-25 mg/ml.

19. The syringe comprising the injectable viscoelastic gel formulation comprising photocrosslinked hyaluronic acid hydrogel according to claim 13, wherein the photocrosslinked hyaluronic acid hydrogel is at a concentration of 10-25 mg/ml.

20. The syringe comprising the injectable viscoelastic gel formulation comprising photocrosslinked hyaluronic acid hydrogel according to claim 14, wherein the photocrosslinked hyaluronic acid hydrogel is at a concentration of 10-25 mg/ml.
